(19) United States
(12) Patent Application Publication
(10) Pub. No.: US 2009/0191277 A1
(43) Pub. Date: Jul. 30, 2009

(54) PROTEIN NANOPARTICLES

(76) Inventor: Makiko AIMI, Kanagawa (JP)

Correspondence Address: BIRCH STEWART KOLASCH & BIRCH, PO BOX 747, FALLS CHURCH, VA, 22040-0747, US

(21) Appl. No.: 12/360,583

(22) Filed: Jan. 27, 2009

(30) Foreign Application Priority Data
Jan. 28, 2008 (JP) 2008-016146

(51) Int. Cl. A61K 9/14; A61K 31/495

(52) U.S. Cl. 424/499; 514/275; 977/773; 977/906

(57) ABSTRACT

It is an object of the present invention to provide a highly safe composition comprising minoxidil and having high transparency due to the small particle size and high permeability into scalp and hair follicles. The present invention provides a protein nanoparticle which comprises minoxidil.

### TECHNICAL FIELD

The present invention relates to particles containing minoxidil.

### BACKGROUND ART

Along with the advancement of studies of drug delivery systems (DDSs), many transdermal therapeutic systems (TTSs) that are intended to have whole-body applications have been developed in recent years. This has followed the development of therapeutic agents such as nitroglycerine and isosorbide nitrate for angina pectoris and therapeutic agents such as scopolamine for motion sickness. TTS products that can be retained for long hours at effective concentrations in blood have been developed. Examples thereof include: an estradiol TTS that is a hormone replacement therapy agent for menopausal disorders and has promising medicinal effects when it is applied once every two days; and a TTS comprising tulobuterol hydrochloride that is an anti asthma agent. In addition, in the field of urology, the clinical development of a transdermal therapeutic system has been attempted, such system comprising oxybutynin, which is a widely used oral therapeutic agent for dysuria. These TTSs have many advantages and thus there are high expectations that they will serve as pharmaceutical products that can improve patient QOL (Yuichiro Nakada et al., Strategies for developing transdermally/transnasally/transpulmonarily absorbable pharmaceutical products (Johokiko Co. Ltd.), P27 (2005)).

Meanwhile, extensive applications of fine particle materials have been expected fer biotechtiology. In particular, the application of nanoparticle materials generated based on the advancement of nanotechnology to food, cosmetics, pharmaceutical products and the like has been actively discussed. In this regard, the results of many studies have been reported.

For instance, regarding cosmetics, more obvious skin-improving effects have been required in recent years. Manufactures have been attempting to improve the functionality and usability of their own products and to differentiate their own products from competitive products by applying a variety of new technologies such as nanotechnology. In general, the stratum corneum layer serves as a barrier for the skin. Thus, medicines are unlikely to permeate therethrough into the skin. In order to obtain sufficient skin-improving effects, it is essential to improve the skin permeability of active ingredients. In addition, it is difficult to formulate many active ingredients due to poor preservation stability or tendency to result in skin irritancy, even if they are highly effective to the skin. In order to solve the above problems, a variety of fine particle materials have been under development for the improvement of transdermal absorption and preservation stability, reduction of skin irritancy, and the like. Recently, a variety of fine particle materials such as ultrafine emulsions and liposomes have been studied (e.g., Mitsuhiro Nishida, Fragrance Journal, November, 17 (2005)). Parfums Christian Dior has succeeded in producing fine liposomes of 0.1 μm in size with soy lecithin. An obtained product called “Capture” contains collagen, elastin, and hyaluronic acid. This beauty essence penetrates the stratum corneum layer (20%) and the dermis layer (48%), and it is assimilated into the fibroblast membrane of the dermis layer. Then, active ingredients are incorporated into cells so as to promote cellular regeneration (e.g., Shukan Shogyo, vol. 1649 (1986)). In the field of hair growth, liposomes have been developed for the purpose of safely delivering effective substances contained therein to hair follicles for the enhancement of the effects of such substances. Further, there have been studies to improve such liposomes (Follicular liposomal delivery systems, J Liposome Res. 2002, 12:143-8).

However, emulsion membranes are physicochemically very weak and unstable. Therefore, emulsion membranes become damaged through contamination with organic or inorganic salts and/or charged substances. In addition, they are very susceptible to heat and light, and thus they are unstable during long-term preservation, which is disadvantageous.
In view of material structure, it is predicted that preservation stability and in vivo particle stability would be significantly improved with the use of a polymeric material instead of an emulsified product or liposomes. However, most studies have involved the use of synthetic polymers mainly obtained via emulsion polymerization. Therefore, it has been necessary to obtain safer carriers. For instance, JP Patent Publication (Kokai) No. 2002-308728 A suggests a transdermally absorbable nanoparticle made from a polymeric material. JP Patent Publication (Kokai) No. 2004-244420 A suggests a cross-linked polymer nanoparticle containing skin care components. These are polymerized products, such as an emulsified product comprising a surfactant and a monomer or macromer (a synthetic polymer having polymerizable groups), which are problematic in terms of safety.

Hiroyuki Tsujimoto, Drug Delivery System, 21-4, 405 (2006) suggests a poly(lactic-co-glycolic acid) (PLGA) nanoparticle that is a biocompatible polymer. However, PLGA is likely to be hydrolyzed and thus it is problematic in terms of preservation stability. In addition, in vivo hydrolysis causes lactic acid production. This might result in adverse effects.

As an aside, minoxidil that has the chemical name “6-(1-piperidinyl)-2,4-pyridinediamine-3-oxide” is known to be applicable to a hair growth agent (see U.S. Pat. No. 4,139,619). Minoxidil is poorly soluble, and thus such an agent usually contains ethanol (50% or more) for dissolving the minoxidil. In this regard, there is a concern about the adverse effects of ethanol. JP Patent Publication (Kokai) No. 2006-176447 A suggests a composition that alleviates the scalp irritation caused by ethanol. However, in such case, the ethanol content is still 40% or more. Thus, this suggestion cannot be a fundamental solution. In addition, minoxidil itself functions as a skin irritant to a slight extent. The use of a hair growth agent containing minoxidil might cause light skin inflammation in rare cases. Therefore, there is a demand for alleviation of skin irritation as described above. Further, an increase in the amount of a solvent in which minoxidil is dissolved causes deterioration in the sensation from use, resulting in stickiness and the like. This has been also problematic.

### DISCLOSURE OF THE INVENTION

It is an object of the present invention to solve the above problems of the prior art. Specifically, it is an object of the present invention to provide a highly safe composition comprising minoxidil and having high transparency due to the small particle size and high permeability into scalp and hair follicles.

As a result of intensive studies in order to achieve the above object, the present inventors demonstrated that protein nanoparticles comprising minoxidil which was prepared by the present inventors are highly safe and have high transparency and favorable permeability into scalp and hair follicles, when applied to the skin. The present invention has been completed based on the above findings.

The present invention provides a protein nanoparticle which comprises minoxidil.

Preferably, the average particle size is 10 to 1000 nm.

Preferably, the protein nanoparticle of the present invention comprises minoxidil in a weight that is 0.01% to 100% of the protein weight.

Preferably, the protein nanoparticle of the present invention further comprises at least one physiologically active ingredient selected from the group consisting of cosmetic ingredients, ingredients for quasi drugs, and ingredients for pharmaceutical products.

Preferably, the protein is at least one selected from the group consisting of collagen, gelatin, acid-treated gelatin, albumin, ovalbumin, casein, transferrin, globulin, fibroin, fibrin, laminin, fibronectin, and vitronectin.

Preferably, the protein is subjected to crosslinking treatment during and/or after nanoparticle formation.

Preferably, an enzyme is used for crosslinking treatment.

Further, the present invention provides a water dispersion product which comprises the protein nanoparticle of the present invention.

Further, the present invention provides a casein nanoparticle prepared by the following steps (a) to (c):
(a) mixing casein with a basic aqueous medium at a pH of 8 or more;
(b) adding minoxidil to the solution obtained in step (a); and
(c) injecting the solution obtained in step (b) into an acidic aqueous medium at a pH of 3.5 to 7.5:

Further, the present invention provides a casein nanoparticle prepared by the following steps (a) to (c):
(a) mixing casein with a basic aqueous medium at a pH of 8 or more;
(b) adding minoxidil to the solution obtained in step (a); and
(c) lowering the pH of the solution obtained in step (b) to a pH value which is different from the isoelectric point by 1 or more units, while stirring the solution.

The protein nanoparticle which comprises minoxidil according to the present invention is a nanoparticle, and thus it is highly absorbable. In addition, according to the present invention, since protein nanoparticles are used, there is no need to use chemical crosslinking agents or synthetic surfactants upon production, which is highly safe. Moreover, hydrophobic minoxidil can be dispersed in a nanoparticle. Accordingly, there is no need to add ethanol in large amounts and thus scalp irritancy caused by ethanol can be substantially prevented.

### BEST MODE FOR CARRYING OUT THE INVENTION

Hereinafter, embodiments of the present invention are described in more detail.

The protein nanoparticle of the present invention is characterized in that it conprises minoxidil. Minoxidil is a compound represented by the generic name “2,4-diamino-6-piperidinopyrimidine-3-oxide” that has been used as an agent for treating hypertension. Then, it has been elucidated that the minoxidil has hair regrowth effects and thus it is used as an ingredient for a hair regrowth agent/hair growth agent.

Types of physiologically active ingredients other than minoxidil used in the present invention are not particularly limited as long as such ingredients are absorbable through the skin so as to exhibit activities. For instance, they can be selected from the group consisting of cosmetic ingredients, ingredients for quasi drugs, and ingredients for pharmaceutical products. Examples thereof include moisturizing agents, whitening agents, hair growth agents, hair nutritional agents, hair regrowth agents, blood circulation promoters, anti-gray hair agents, anti-aging agents, antioxidants, collagen synthesis promoters, anti-wrinkle agents, anti-acne agents, vitamins, ultraviolet absorbing agents, aroma chemicals, coloring agents, antiperspirants, cooling agents, warming agents, melanogenesis inhibitors, melanocyte activators, antibiotics, carcinostatic agents, anti-inflammatory agents, antiallergic agents, hormonal agents, antithrombotic agents, immunosuppressants, skin disease treatment agents, antifungal agents, nucleic acid drugs, anesthetic agents, antipyretics, analgesic agents, antipruritic agents, anti-edema agents, hypnosedatives, antianxiety agents, stimulants, psychoneurotic agents, muscular relaxants, antidepressants, combination remedies for common cold, autonomic agents, antispasmodic agents, diaphoretics, anti-sweating agents, cardiotonic agents, agents used for arrhythmia, antiarrhythmic agents, vasoconstrictors, vasodilators, antiarrhythmic agents, antihypertensive agents, diabetic treatment agents, agents used for hyperlipidemia, respiratory stimulants, antitussives, vitamins, agents used for parasitic skin diseases, homeostatic agents, polypeptides, hormones, parakeratosis inhibitors, vaccines, and skin softeners. The above physiologically active ingredients may be used alone or in combinations of two or more.

Types of hair growth agents, hair nutritional agents, hair regrowth agents other than minoxidil used in the present invention are not particularly limited. However, specific examples thereof include: glycyrrhetic acid or derivatives thereof; glycyrrhizic acid or derivatives thereof; hinokitiol, vitamin E or derivatives thereof; vitamin C and derivatives thereof; vitamin B3 derivatives such as nicotinic acid benzyl, nicotinic acid tocopherol, nicotinic acid β-butoxy ester, and nicotinamide; vitamin B5 and derivatives thereof such as pantothenyl ethyl ether and pantothenyl alcohol; carotenoids such as astaxanthin and β-carotene; isopropyl methylphenol; cephalathin; ethynyl estradiol; diphenhydramine hydrochloride; menthol; 6-benzyl aminopurine; pentadecanoic acid and derivatives thereof; t-flavanone; adenosine and derivatives thereof, carpronium chloride; finasteride; plant extracts such as Swertia japonica extract, Sophorae Radix (sophora root) extract, licorice extract, Lepisorus thunbergianus extract, capsicum extract, Ampelopsis cantoniensis var. grossedentata extract, carrot extract, Taraxacum mongolicum Hand.-Mazz. extract, tree peony extract, and mandarin orange extract. The above hair growth agents, hair nutritional agents, hair regrowth agents may be used alone or in combinations of two or more.

Examples of blood circulation promoters include nicotinic acid, Swertia japonica extract, γ-oxazole, alkoxycarbonylpyridine N-oxide, carpronium chloride, and acetylcholine or derivatives thereof.

Examples of anti-inflammatory agents include: compounds and salts and derivatives thereof selected from the group consisting of azulene, allantoin, lysozyme chloride, guaiazulene, diphenhydramine hydrochloride, hydrocortisone acetate, predonisolone, glutathione, saponin, methyl salicylate, mefenamic acid, phenylbutazone, indomethacin, ibuprofen, and ketoprofen; and extracts such as Scutellariae radix extract, Artemisia capillaris extract, balloon flower (Platycodon grandiflorus) extract, Armeniacae semen extract, gardenia extract, Sasa veitchii extract, gentiana extract, comfrey extract, white birch extract, mallow extract, Persicae semen extract, peach leaf extract, and Eriobotryae folium extract.

Examples of moisturizing agents include hyaluronic acid, ceramide, Lipidure, isoflavone, amino acid, collagen, mucopolysaccharide, fucoidan, lactoferrin, sorbitol, chitin/chitosan, malic acid, glucuronic acid, placenta extract, seaweed extract, moutan cortex extract, sweet tea extract, hypericum extract, coleus extract, Euonymus japonicus extract, safflower extract, Rosa rugosa flower extract, Polyporus sclerotium extract, hawthorn extract, rosemary extract, duke extract, chamomile extract, Lamium album extract, Litchi Chinensis extract, Achillea millefolium extract, aloe extract, marronnier extract, Thujopsis dolabrata extract, Fucus extract, Osmoin extract, oat bran extract, tuberosa polysaccharide, Cordyceps sinensis (plant worm) extract, barley extract, orange extract, Rehmannia glutinosa extract, zanthoxylum extract, and Coix lachryma-jobi extract.

The protein nanoparticle of the present invention contains minoxidil in a weight that is preferably 0.1% to 100% of the protein weight and further preferably 0.1% to 50% of the protein weight

A composition comprising the protein nanoparticles of the present invention may be prepared. The composition preferably 0.01% to 50% by weight and most preferably 0.1% to 10% by weight protein nanoparticles.

According to the present invention, minoxidil may be added during or after protein nanoparticle formation.

The average particle size of protein nanoparticles used in the present invention is generally 1 to 1000 nm, preferably 10 to 1000 nm, more preferably 10 to 200 nm, further preferably 10 to 100 nm, and particularly preferably 20 to 50 nm.

The type of protein used in the present invention is not particularly limited. However, a protein having a lysine residue and a glutamine residue is preferable. In addition, such protein having a molecular weight of approximately 10,000 to 1,000,000 is preferably used. The origin of the protein is not particularly limited. However, a human-derived protein is preferably used. Specific examples of a protein that can be used include, but are not limited to, the following compounds according to the present invention: at least one selected from the group consisting of collagen, gelatin, acid-treated gelatin, albumin, ovalbumin, casein, transferrin, globulin, fibroin, fibrin, laminin, fibronectin, and vitronectin. In addition, the origin of the protein is not particularly limited. Thus, any bovine, swine, or fish protein, as well as recombinant protein of any thereof, can be used. Examples of recombinant gelatin that can be used include, but are not limited to, gelatins described in EP1014176 A2 and U.S. Pat. No. 6,992,172. Among them, casein, acid-treated gelatin, collagen, or albumin is preferable. Further, casein or acid-treated gelatin is most preferable. Upon the use of casein according to the present invention, the origin of the casein is not particularly limited. Casein may be milk-derived or bean-derived. Any of α-casein, β-casein, γ-casein, and κ-casein, as well as a mixture of any thereof, can be used. Caseins may be used alone or in combinations of two or more.

Proteins used in the present invention may be used alone or in combinations of two or more.

According to the present invention, it is possible to carry out a crosslinking treatment for a protein during and/or after nanoparticle formation. For the crosslinking treatment, an enzyme can be used. Any enzyme may be used without particular limitation as long as it has been known to have the effect of causing protein crosslinking. Among such enzymes, transglutaminase is preferable.

Transglutaminase may be derived from a mammal or a microorganism. A recombinant transglutaminase can be used. Specific examples thereof include the Activa series by Ajinomoto Co., Inc., commercially available mammalian-derived transglutaminase serving as a reagent, such as guinea pig liver-derived transglutaminase, goat-derived transglutaminase, rabbit-derived transglutaminase, or human-derived recombinant transglutaminase produced by, for example, Oriental Yeast Co., Ltd., Upstate USA Inc., and Biodesign International.

The amount of an enzyme used in a crosslinking treatment according the present invention can be adequately determined depending upon protein type. In general, an enzyme can be added in a weight that is 0.1% to 100% and preferably approximately 1% to 50% of the protein weight.

The duration for an enzymatic crosslinking reaction can be adequately determined depending upon protein type and nanoparticle size. However, in general, the reaction can be carried out for 1 to 72 and preferably 2 to 24 hours.

The temperature for an enzymatic crosslinking reaction can be adequately determined depending upon protein type and nanoparticle size. In general, the reaction can be carried out at 0° C. to 80° C. and preferably at 25° C. to 60° C.

Enzymes used in the present invention may be used alone or in combinations of two or more.

Nanoparticles of the present invention can be prepared in accordance with Patent Document: JP Patent Publication (Kokai) No. 6-79168 A (1994); or C. Coester, Journal Microcapsulation, 2000, vol. 17, pp. 187-193, provided that an enzyme is preferably used instead of glutaraldehyde for a crosslinking method.

In addition, according to the present invention, the enzymatic crosslinking treatment is preferably carried out in an organic solvent. The organic solvent used herein is preferably an aqueous organic solvent such as ethanol, isopropanol, acetone, or THF.

Further, according to the present invention, it is preferable to remove an organic solvent by distillation subsequent to a crosslinking treatment, followed by water dispersion. It is also possible to add water prior to or subsequent to removal of an organic solvent by distillation.

It is also possible to add at least one component selected from the group consisting of lipids (e.g., phospholipid), anionic polysaccharides, cationic polysaccharides, anionic proteins, cationic proteins, and cyclodextrin to the composition for hair of the present invention. The amounts of lipid (e.g. phospholipid), anionic polysaccharide, cationic polysaccharide, anionic protein, cationic protein, and cyclodextrin to be added are not particularly limited. However, they can be added usually in a weight that is 0.1% to 100% of the protein weight. In the case of the composition for hair of the present invention, it is possible to adjust the release rate by changing the ratio of the above components to the protein.

Specific examples of phospholipids that can be used in the present invention include, but are not limited to, the following compounds according to the present invention: phosphatidylcholine (lecithin), phosphatidylethanolamine, phosphatidylserine, phosphatidylinositol, phosphatidylglycerol, diphosphatidylglycerol, and sphingomyelin.

Anionic polysaccharides that can be used in the present invention are polysaccharides having an acidic polar group such as a carboxyl group, a sulfate group, or a phosphate group. Specific examples thereof include, but are not limited to, the following compounds according to the present invention: chondroitin sulfate, dextran sulfate, carboxymethyl cellulose, carboxymethyl dextran, alginic acid, pectin, carrageenan, fucoidan, agaropectin, porphyran, karaya gum, gellan gum, xanthan gum, and hyaluronic acids.

Cationic polysaccharides that can be used in the present invention are polysaccharides having a basic polar group such as an amino group. Examples thereof include, but are not limited to, the following compounds according to the present invention: polysaccharides such as chitin or chitosan, which comprise, as a monosaccharide unit, glucosamine or galactosamine.

Anionic proteins that can be used in the present invention are proteins and lipoproteins having a more basic isoelectric point than the physiological pH. Specific examples thereof include, but are not limited to, the following compounds according to the present invention: poly glutamic acid, polyaspartic acid, lysozyme, cytochrome C, ribonuclease, trypsinogen, chymotrypsinogen, and α-chymotrypsin.

Cationic proteins that can be used in the present invention are proteins and lipoproteins having a more acidic isoelectric point than the physiological pH. Specific examples of such cationic protein include, but are not limited to, the following compounds according to the present invention: polylysine, polyarginine, histone, protamine, and ovalbumin.

According to the present invention, it is possible to use casein nanoparticles prepared by the following steps (a) to (c):
(a) mixing casein with a basic aqueous medium at a pH of 8 or more;
(b) adding minoxidil to the solution obtained in step (a); and
(c) injecting the solution obtained in step (b) into an acidic aqueous medium at a pH of 3.5 to 7.5:

According to the present invention, it is possible to use casein nanoparticles prepared by the following steps (a) to (c):
(a) mixing casein with a basic aqueous medium at a pH of 8 or more;
(b) adding minoxidil to the solution obtained in step (a); and
(c) lowering the pH of the solution obtained in step (b) to a pH value which is different from the isoelectric point by 1 or more units, while stirring the solution.

According to the present invention, it is possible to prepare casein nanoparticles of desired sizes. Also, with the use of interaction between a hydrophobic active ingredient for hair and a casein hydrophobic domain, it is possible for casein nanoparticles to contain minoxidil. In addition, it was found that such particles remain stable in an aqueous solution.

The method for preparing casein nanoparticles of the present invention involves a method wherein casein is mixed with a basic aqueous medium solution and the solution is injected into an acidic aqueous medium, and a method wherein casein is mixed with a basic aqueous medium and the pH of the medium is lowered during stirring, for example.

The method wherein casein is mixed with a basic aqueous medium solution and the solution is injected into an acidic aqueous medium is preferably carried out using a syringe for convenience. However, there is no particular limitation as long as the injection rate, solubility, temperature, and stirring conditions are satisfied. Injection can be carried out usually at an injection rate of 1 mL/min to 100 mL/min. The temperature of the basic aqueous medium can be adequately determined. In general, the temperature is 0° C. to 80° C. and preferably 25° C. to 70° C. The temperature of an aqueous medium can be adequately determined. In general, the temperature can be 0° C. to 80° C. and preferably 25° C. to 60° C. The stirring rate can be adequately determined. However, in general, the stirring rate can be 100 rpm to 3000 rpm and preferably 200 rpm to 2000 rpm.

In the method wherein casein is mixed with a basic aqueous medium and the pH of the medium is lowered during stirring, it is preferable to add acid dropwise for convenience. However, there is no particular limitation as long as solubility, temperature, and stirring conditions are satisfied. The temperature of a basic aqueous medium can be adequately determined. However, in general, the temperature can be 0° C. co 80° C. and preferably 25° C. to 70° C. The stirring rate can be adequately determined. However, in general, the stirring rate can be 100 rpm to 3000 rpm and preferably 200 rpm to 2000 rpm.

The aqueous medium that can be used for the present invention is an aqueous solution or a buffer comprising an organic acid or base or an inorganic acid or base.

Specific examples thereof include, but are not limited to, aqueous solutions comprising: organic acids such as citric acid, ascorbic acid, gluconic acid, carboxylic acid, tartaric acid, succinic acid, acetic acid, phthalic acid, trifluoroacetic acid, morpholinoethanesulfonic acid, and 2-[4-(2-hydroxyethyl)-1-piperazinyl]ethanesulfonic acid; organic bases such as tris (hydroxymethyl), aminomethane, and ammonia; inorganic acids such as hydrochloric acid, perchloric acid, and carbonic acid; and inorganic bases such as sodium phosphate, potassium phosphate, calcium hydroxide, sodium hydroxide, potassium hydroxide, and magnesium hydroxide.

The concentration of an aqueous medium used in the present invention is preferably approximately 10 mM to 1 M, and more preferably approximately 20 mM to 200 mM.

The pH of a basic aqueous medium used in the present invention is preferably 8 or more, more preferably 8 to 12, and further preferably 10 to 12. When the pH is excessively high, there is concern regarding hydrolysis or risks in handling. Thus, the pH is preferably in the above range.

According to the present invention, the temperature at which casein is mixed with a basic aqueous medium at a pH of 8 or more is preferably 0° C. to 80° C., more preferably 10° C. to 60° C., and further preferably 20° C. to 40° C.

The pH of an acidic aqueous medium used in the present invention is preferably 3.5 to 7.5 and more preferably 5 to 6. When the pH does not fall in the above range, the particle size tends to become large.

The composition comprising the protein nanoparticles of the present invention may further comprise an additive. Examples of an additive that can be used include, but are not limited to, at least one selected from the group consisting of moisturizing agents, softening agents, transdermal absorption enhancers, soothing agents, preservatives, antioxidants, coloring agents, thickeners, aroma chemicals, and pH adjusters.

Specific examples of moisturizing agents that can be used in the present invention include, but nct limited to, the following compounds according to the present invention agar, diglycerin, distearyldimonium hectorite, butylene glycol, polyethylene glycol, propylene glycol, hexylene glycol, Coix lachryma-jobi extract, vaseline, urea, hyaluronic acid, ceramide, Lipidure, isoflavone, amino acid, collagen, mucopolysaccharide, fucoidan, lactoferrin, sorbitol, chitin/chitosan, malic acid, glucuronic acid, placenta extract, seaweed extract, moutan cortex extract, sweet tea extract, hypericum extract, coleus extract, Euonymus japonicus extract, safflower extract, Rosa rugosa flower extract, Polyporus sclerotium extract, hawthorn extract, rosemary extract, duke extract, chamomile extract, Lamium album extract, Litchi Chinensis extract, Achillea millefolium extract, aloe extract, marronnier extract, Thujopsis dolabrata extract, Fucus extract, Osmoin extract, oat bran extract, tuberosa polysaccharide, Cordyceps sinensis (plant worm) extract, barley extract, orange extract, Rehmannia glutinosa extract, zanthoxylumb extract, and Coix lachryma-jobi extract.

Specific examples of softening agents that can be used in the present invention include, but are not limited to, the following compounds according to the present invention: glycerin, mineral oil, and emollient ingredients (e.g., isopropyl isostearate, polyglyceryl isostearate, isotridecyl isononanoate, octyl isononanoate, oleic acid, glyceryl oleate, cocoa butter, cholesterol, mixed fatty acid triglyceride, dioctyl succinate, suc;ose tetrastearate triacetate, cyclopentasiloxane, sucrose distearate, palmitateoctyl, oacyl hydroxystearate, arachidyl behenate, sucrose polybehenate, polyrmethylsilsesquioxane, myristyl alcohol, cetyl myristate, myristyl myristate, and hexyl laurate).

Specific examples of transdermal absorption enhancers that can be used in the present invention include, but are not limited to, the following compounds according to the present invention: ethanol, isopropyl myristate, citric acid, squalane, oleic acid, menthol, limonene, N-methyl-2-pyrrolidone, diethyl adipate, diisopropyl adipate, diethyl sebacate, diisopropyl sebacate, isopropyl palmitate, isopropyl oleate, octyldodecyl oleate, isostearyl alcohol, 2-octyldodecanol, urea, vegetable oil, and animal oil.

Specific examples of soothing agents that can be used in the present invention include, but are not limited to, the following compounds according to the present invention: benzyl alcohol, procaine hydrochloride, xylocaine hydrochloride, and chlorobutanol.

Specific examples of preservatives that can be used in the present invention include, but are not limited to, the following compounds according to the present invention: benzoic acid, sodium benzoate, paraben, ethylparaben, methylparaben, propylparaben, butylparaben, potassium sorbate, sodium sorbate, sorbic acid, sodium debydroacetate, hydrogen peroxide, formic acid, ethyl formate, sodium hypochlorite, propionic acid, sodium propionate, calcium propionate, pectin degradation products, polylysine, phenol, isopropylmethyl phenol, orthophenylphenol, phenoxyethanol, resorcin, thymol, thiram, and tea tree oil.

Specific examples of antioxidants that can be used in the present invention include, but are not limited to, the following compounds according to the present invention: vitamin A, retinoic acid, retinol, retinol acetate, retinol palmitate, retinyl acetate, retinyl palmitate, tocopheryl retinoate, vitamin C and derivatives thereof, kinetin, β-carotene, astaxanthin, lutein, lycopene, tretinoin, vitamin E, a-lipoic acid, coenzyme Q10, polyphenol, SOD, and phytic acid.

Specific examples of coloring agents that can be used in the present invention but are not limited to, the following compounds according to the present invention: kill pigment, orange dye, cacao dye, kaoline, carmines, ultramarine blue, cochineal dye, chrome oxide, iron oxide, titanium dioxide, tar dye, chlorophyll, and legal dyes that can be used for cosmetic ingredients, ingredients for quasi drugs, and ingredients for pharmaceutical products.

Specific examples of thickeners that can be used in the present invention include, but are not limited to, the following compounds according to the present invention: quince seed, carrageenan, gum arabic, karaya gum, xanthan gum, gellan gum, tamarind gum, locust bean gum, gum traganth, pectin, starch, cyclodextrin, methylcellulose, ethylcellulose, carboxymethylcellulose, sodium alginate, polyvinyl alcohol, polyvinyl pyrrolidone, carboxyvinyl polymer, and sodium polyacrylate.

Specific examples of aroma chemicals that can be used in the present invention include, but are not limited to, the following compounds according to the present invention: musk, acacia oil, anise oil, ylang ylang oil, cinnamon oil, jasmine oil, sweet orange oil, spearmint oil, geranium oil, thyme oil, neroli oil, mentha oil, hinoki (Japanese cypress) oil, fennel oil, peppermint oil, bergamot oil, lime oil, lavender oil, lemon oil, lemongrass oil, rose oil, rosewood oil, anisaldehyde, geraniol, citral, civetone, muscone, limonene, and vanillin.

Specific examples of pH adjusters that can be used in the present invention include, but are not limited to, the following compounds according to the present invention: sodium citrate, sodium acetate, sodium hydroxide, potassium hydroxide, phosphoric acid, and succinic acid.

The dosage form of a composition comprising the protein nanoparticle of the present invention is not particularly limited. However, examples thereof include liquid formulations for external use, fomentations, embrocations, bathing agents, bath additives, disinfectants, ointments, gels, creams, pastes, adhesive skin patches, plasters, wound-surface-covering agents, wound-surface-covering gauzes, hemostatics, adhesives, adhesive tape, adhesive tape for transdermal absorption, wound protective agents, aerosols, lotions, tonics, liniments, emulsions, suspensions, saturants, tinctures, powders, foaming agents, skin lotions, massage creams, nourishing creams, face packs, sheet-type drugs for external use, cosmetics for makeup, skin coloring agents for external use, cosmetic skin adhesives, shampoos, rinses, permanent wave compositions, hair dyes, body soap, soap, bath agents, sun care products (e.g., sunscreens, sun tanning oils, and after-sun lotions), and fragrances.

The protein nanoparticle of the present invention can be administered transdermally or transmucosally.

The dose of the protein nanoparticle of the present invention can be adequately determined depending upon amount of minoxidil used, patient weight, and disease conditions, for example. The dose for single administration can be generally approximately 1 μg to 50 mg/cm² and preferably approximately 2.5 μg to 10 mg/cm².

The present invention is hereafter described in greater detail with reference to the following examples, although the technical scope of the present invention is not limited thereto.

### EXAMPLES

### Example 1

Milk-derived casein Na (10 mg; Wako Pure Chemical Industries, Ltd.) was mixed with 50 mM phosphate buffer (pH 9, 1 mL). The casein solution (1 ml) was injected into 200 mM phosphate buffer water (pH 5, 10 mL) in which minoxidil (1.7 mg) had been dissolved with the use of a microsyringe at an external temperature of 40° C. during stirring at 800 rpm. Thus, a water dispersion of casein nanoparticles containing minoxidil was obtained. The average particle size of the above particles was measured with a “Microtrac” light scattering photometer (NIKKISO Co., Ltd.) and found to be 55 nm.

### Example 2

Milk-derived casein Na (10 mg; Wako Pure Chemical Industries, Ltd.) and minoxidil (1 mg) were mixed with 50 mM phosphate buffer (pH 10, 1 mL). Hydrochloric acid was added thereto so that the pH was adjusted to 7. Thus, casein nanoparticles were obtained.

The average particle size of the above particles was measured with a “Nano-ZS” light scattering photometer (Malvern Instruments Ltd) and found to be 23 nm.

### Example 3

Milk-derived casein Na (10 mg; Wako Pure Chemical Industries, Ltd.) and minoxidil (1 mg) were mixed with 100 mM phosphate buffer (pH 10, 1 mL). Glycyrrhetic acid (3.4 mg; Wako Pure Chemical Industries, Ltd.) was dissolved in ethhanol (0.1 mL). These two different solutions were mixed together. Hydrochloric acid was added thereto so that the pH was adjusted to 7. Thus, casein nanoparticles were obtained.

The average particle size of the above particles was measured with a “Nanotrac” light scattering photometer (NIKKISO Co., Ltd.) and found to be 22 nm.

### Example 4

Milk-derived casein Na (20 mg; Wako Pure Chemical Industries, Ltd.) and minoxidil (1 mg) were mixed with 100 mM phosphate buffer (pH 10, 1 mL). Hinokitiol (1.7 mg; Wako Pure Chemical Industries, Ltd.) was dissolved in ethanol (0.25 mL). These two different solutions were mixed together. Hydrochloric acid was added thereto so that the pH was adjusted to 7. Thus, casein nanoparticles were obtained. The average particle size of the above particles was measured with a “Nanotrac” light scattering photometer (NIKKISO Co., Ltd.) and found to be 21 nm.

### Example 5

Milk-derived casein (100 mg; Wako Pure Chemical Industries, Ltd.) and minoxidil (0.5 mg) were mixed with 50 mM phosphate buffer (pH 10, 10 mL). FinaFteride (1 mg; LKT Labs, Inc) was dissolved in ethanol (50 μL). These two different solutions were mixed together. Hydrochloric acid was added thereto so that the pH was adjusted to 7. Thus, casein nanoparticles were obtained.

The average particle size of the above particles was measured with a “Nanotrac” light scattering photometer (NIKKISO Co., Ltd.) and found to be 22 nm.

### Example 6

Acid-treated gelatin (10 mg) and TG-S (5 mg; Ajinomoto Co., Inc.) were dissolved in water (1 mL). The gelatin solution (1 ml) was injected into ethanol (10 mL) in which minoxidil (1.7 mg) had been dissolved with the use of a microsyringe at an external temperature of 40° C. during stirring at 800 rpm. Thus, gelatin nanoparticles were obtained. The gelatin nanoparticles were allowed to stand at an external temperature of 55° C. for 5 hours for enzymatic crosslinking. The average particle size of the above particles was measured with a “Microtrac” light scattering photometer (NIKKISO Co., Ltd.) and found to be 80 nm.

Water (5 mL) was added to the obtained gelatin nanoparticle dispersion and ethanol was removed therefrom by means of a rotary evaporator. Thus, a water dispersion of gelatin nanoparticles was obtained.

The average particle size of the above particles was measured with a “Microtrac” light scattering photometer (NIKKISO Co., Ltd.) and found to be 211 nm.

### Test Example 1

The dispersions of nanoparticles containing hair growth agents described in Examples 2 to 6 were preserved at room temperature for 1 month. Thereafter, average particle size was measured using a Microtrac (NIKKISO Co., Ltd.).

As Comparative example 1, a “NanoImpact” synthetic polymer (PLGA) nanoparticle dispersion (Hosokawa Micron Corporation) was used.

Table 1 shows measurement results obtained in Experimental example 1. Sedimentation was observed in Comparative Example 1.

TABLE 1
Experimental example 1

| | Comparative | | | | | | |
|---|---|---|---|---|---|---|---|
| Appearance | Example 1 | Example 1 | Example 2 | Example 3 | Example 4 | Example 5 | Example 6 |
| When | 600 nm | 55 nm | 23 nm | 22 nm | 21 nm | 22 nm | 211 nm |
| prepared | | | | | | | |
| 1 month | N.D. | 80 nm | 24 nm | 20 nm | 23 nm | 23 nm | 220 nm |
| later | | | | | | | |

Five trial volunteers evaluated the water dispersion products of the above Examples in terms of the sensation from use. Good results expressed as “ease of application,” “excellent freshened sense,” “no stickiness,” and the like were obtained in relation to the sensation from use.

Based on the Examples, it is understood that minoxidil can be stably maintained in an aqueous product that does not contain a solvent such as ethanol in the case of the constitution of the present invention. Therefore, high safety and high transparency due to the small particle size can be realized. In addition, the use of natural polymers contributes to high safety.

1. A protein nanoparticle which comprises minoxidil.

2. The protein nanoparticle of claim 1, wherein the average particle size is 10 to 1000 nm.

3. The protein nanoparticle of claim 1, which comprises minoxidil in a weight that is 0.01% to 100% of the protein weight.

4. The protein nanoparticle of claim 1, which further comprises at least one physiologically active ingredient selected from the group consisting of cosmetic ingredients, ingredients for quasi drugs, and ingredients for pharmaceutical products.

5. The protein nanoparticle of claim 1, wherein the protein is at least one selected from the group consisting of collagen, gelatin, acid-treated gelatin, albumin, ovalbumin, casein, transferrin, globulin, fibroin, fibrin, laminin, fibronectin, and vitronectin.

6. The protein nanoparticle according to claim 1, wherein the protein is subjected to crosslinking treatment during and/or after nanoparticle formation.

7. The protein nanoparticle of claim 6, wherein an enzyme is used for crosslinking treatment.

8. A water dispersion product which comprises the protein nanoparticle of claim 1.

9. A casein nanoparticle prepared by the following steps (a) to (c):
(a) mixing casein with a basic aqueous medium at a pH of 8 or more;
(b) adding minoxidil to the solution obtained in step (a); and
(c) injecting the solution obtained in step (b) into an acidic aqueous medium at a pH of 3.5 to 7.5:

10. A casein nanoparticle prepared by the following steps (a) to (c):
(a) mixing casein with a basic aqueous medium at a pH of 8 or more;
(b) adding minoxidil to the solution obtained in step (a); and
(c) lowering the pH of the solution obtained in step (b) to a pH value which is different from the isoelectric point by 1 or more units, while stirring the solution.
